(19) United States
(12) Patent Application Publication
(10) Pub. No.: US 2013/0045133 A1
(43) Pub. Date: Feb. 21, 2013

(54) METHODS AND APPARATUS FOR COMMERCE MEDIA STERILIZATION

(71) Applicant: David Maguire, Burbank, CA (US)

(72) Inventor: David Maguire, Burbank, CA (US)

(73) Assignee: VIALUX SYSTEMS, INC., Burbank, CA (US)

(21) Appl. No.: 13/588,642

(22) Filed: Aug. 17, 2012

Related U.S. Application Data
(60) Provisional application No. 61/525,728, filed on Aug. 20, 2011.

(51) Int. Cl. A61L 2/10; A61L 2/00

(52) U.S. Cl. 422/24; 422/1; 250/455.11

(57) ABSTRACT

The invention relates to methods and apparatus for preventing the contraction of the causative agents of infectious diseases, such as, for example, bacteria and viruses by way of the most common public activities, via the interaction of commerce and the exchange of monetary goods. More specifically, the invention generally relates to methods and apparatus for sterilizing commerce media using the germicidal properties of ultraviolet light and/or ozone.

### CROSS REFERENCE TO RELATED APPLICATIONS

This application claims the benefit under 35 U.S.C. 119(e) of U.S. Provisional Application No. 61/525,728, filed Aug. 20, 2011, entitled “Commerce Media Sterilizer,” which is hereby incorporated herein by reference in its entirety.

### BACKGROUND

This invention relates to an apparatus for sterilizing commerce media such as legal tender, coins, checks, and any and all other conveyances of monetary exchange used and exchanged by the public. In particular, but not by way of limitation, the invention relates to an apparatus which uses germicidal ultraviolet radiation to kill contaminants such as bacteria and viruses on commerce media.

It is well established that many forms of infectious disease, such as the common cold, are contracted by and spread to the general public, primarily by direct hand contact and indirectly by way of intermediate materials.

Furthermore, it is also well known that objects and materials that are freely and continuously transported, transferred, and exchanged in and by the general public can be a source for spreading the causative agents of infectious diseases, such as, for example, bacteria and viruses. Recently, it has been discovered that bacteria continue to live on surfaces and objects in the public space that are used and exchanged by numerous individuals within short periods of time. Similarly disease causing pathogenic agents can live on many different kinds of surfaces and therefore can be a vector for the quick, efficient and expansive spread of various diseases locally, regionally, nationally and internationally within a short span of time.

Accordingly, a need exists to have a convenient, commonly available and pervasively used sterilization method and apparatus for sterilizing commerce media thereby preventing the transmission and acquisition of object-borne disease spreading microorganisms.

### SUMMARY

Embodiments of the invention are shown in the drawings and are summarized below. It is to be understood, however, that there is no intention to limit the invention to the forms described in this Summary of the Invention or in the accompanying Detailed Description. One skilled in the art can recognize that there are numerous modifications, equivalents and alternative constructions that fall within the spirit and scope of the invention as expressed in the claims.

In some embodiments, an apparatus can include an outer casing configured to substantially enclose an inner volume, an inner container configured to receive a paper currency, and an actuator configured to be actuated by the movement of the inner container. The apparatus can further include a sterilizing element disposed within the interior volume and be configured to effectively sterilize the paper currency based on the actuation of the actuator. In some embodiments, a method includes moving a container configured to hold a paper currency from a first position outside of a casing to a second position inside of the casing. The method involves actuating a sterilizing element, based on the movement of the container, which can effectively sterilize the paper currency. The method further involves deactivating the sterilizing element after a pre-determined time period.

In some embodiments, an apparatus includes a case, a drawer movably disposed within the case, a first ultraviolet light source disposed within the case, and a second ultraviolet light source disposed within the case such that the first ultraviolet light source and the second ultraviolet light source are disposed opposite the drawer. The apparatus can further include an actuator operatively coupled to and configured to activate the first ultraviolet light source and the second ultraviolet light source. The apparatus can further include an exhaust fan coupled to the case and is configured to move air from an area inside the case to an area outside the case.

The invention relates to methods and apparatus for minimizing, eliminating and preventing the transfer and contraction of the causative agents of infectious diseases, such as, for example, bacteria and viruses by way of the most common public activities, via the interaction of commerce and the exchange of monetary goods. More specifically, this invention generally relates to, but is not limited to, methods and apparatus for sterilizing commerce media at a key point of exchange, the point of sale (POS), through a POS cash system capable of a germicidal treatment of the cash and coins in exchange. The treatment process is a waterless, chemical free method of utilizing the germicidal properties of ultraviolet light (and optionally, UV created ozone) for inactivating pathogens commonly found on and transferred by way of commerce media, which are placed within the currency drawer of the POS cash system. The invention provides a safe and highly effective means of sterilizing commerce media for the benefit of public safety.

### BRIEF DESCRIPTION OF THE DRAWINGS

FIG. 1 is a schematic illustration of a commerce media sterilizing system according to an embodiment.

FIG. 2 is an example of a flow chart showing a specific method to destroy pathogenic agents on multiple conveyances of monetary exchange via the use of the commerce media sterilizing system, according to a first embodiment.

FIG. 3 is an example of a flow chart showing a specific method to destroy pathogenic agents on multiple conveyances of monetary exchange via the use of the commerce media sterilizing system, according to an embodiment.

FIG. 4 is a block diagram of a point of sale apparatus that includes a commerce media storage and sterilization system, according to an embodiment.

FIG. 5 is an illustration of an example of a point of sale apparatus, according to an embodiment, which can incorporate the commerce media sterilizer.

FIG. 6 is an exploded perspective view of a currency storage and sterilization module of a point of sale apparatus that can implement a method of sterilizing commerce media via the use of UV light and ozone, according to an embodiment.

FIG. 7 is an enlarged cross-sectional view of a currency storage and sterilization module of a point of sale apparatus that can implement a method of sterilizing commerce media via the use of UV light and ozone, according to an embodiment.

FIG. 8 is an example of a flow chart showing methods of activating UV-light sources, according to embodiments.

FIG. 9 is an exploded perspective view of a currency storage and sterilization module of a point of sale apparatus that can implement a method of sterilizing commerce media via the use of UV light, according to an embodiment.

FIG. 10 is an image of an example of a currency sterilization device, according to an embodiment.

### DETAILED DESCRIPTION

In some embodiments, an apparatus can include an outer casing configured to substantially enclose an interior volume, an inner container configured to receive a paper currency, and an actuator configured to be actuated by the movement of the inner container. The apparatus can further include a sterilizing element disposed within the interior volume and configured to effectively sterilize the paper currency based on the actuation of the actuator.

In some embodiments, the apparatus can include an outer container in any form configured to receive an amount of currency therein. In some embodiments, the sterilizing element can include one, or a combination of, ultraviolet light source(s) such as, for example, germicidal ultraviolet-C light (UV-C 200 to 280 nm) light, or ozone producing vacuum ultraviolet light (VUV 100 to 200 nm) or ozone destroying ultraviolet light (UV-C or UV-B 200 to 300 nm). In some embodiments, the sterilizing element can be actuated for a predetermined time period.

The inner container can be, for example, a drawer or other insert configured to hold currency of any form. In some embodiments, the inner container is configured to move from a first configuration (e.g., outside of the outer container) to a second configuration (e.g., inside the outer container) such that the inner container engages an actuator when in the second configuration. In some embodiments, the actuator can be, for example, a catch/release mechanism that can utilize a relay switch to indicate and transmit the configuration state of the inner container with respect to the outer container. The configuration state is transmitted to a processor. In some embodiments, the actuator can be, for example, a catch/release mechanism that can utilize a switch biased to one of a first or a second configuration that can be used to indicate the configuration state of the inner container. In some embodiments, the apparatus can also include an exhaust fan that is configured to move air from within the interior volume to an area outside the POS apparatus.

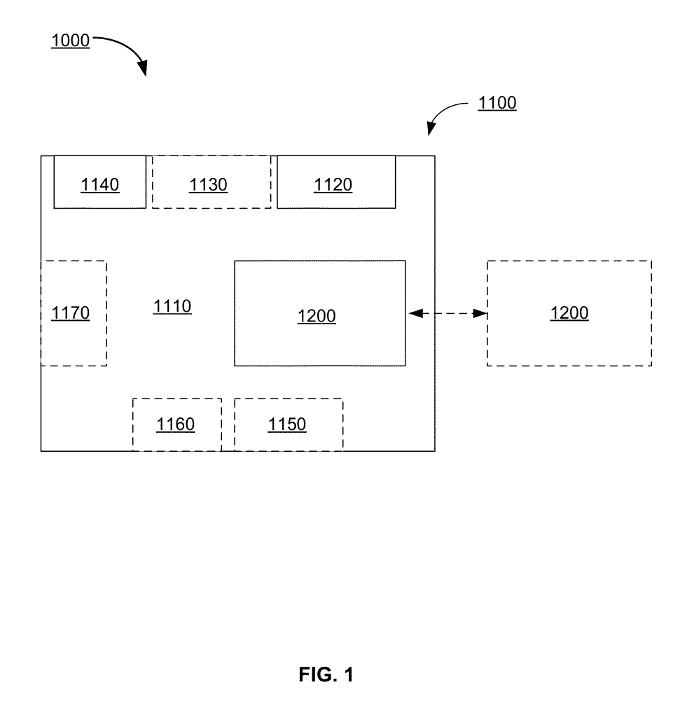

FIG. 1 is a schematic illustration of a commerce media sterilizing system, according to an embodiment. The sterilizing system 1000 includes an outer casing 1100 which substantially encloses an inner volume 1110, and an inner container 1200. The inner container 1200 can be configured to move back and forth from a first configuration outside the casing 1100 to a second configuration inside the casing 1100 where the inner container 1200 can engage the actuators 1140 and/or 1160 in the second configuration. The outer casing 1100 can include a first UV-light source 1120, a first actuator 1140, and optionally, a second UV-light source 1130, a third UV light source 1150, a second actuator 1160, and an air circulation mechanism 1170. The actuators 1140 and/or 1160 are configured to activate the first ultraviolet light source 1120, and/or the second ultraviolet light source 1130, and/or the third ultraviolet light source 1150, and/or the air circulation mechanism 1170.

The air circulation mechanism 1170 includes an exhaust fan coupled to the outer casing 1100 and is configured to move air from within the inner volume 1110 of the outer casing 1100 to an area outside the outer casing 1100. In some embodiments, the air circulation mechanism 1170 is configured to operate during at least a portion of a time period when the first ultraviolet light source 1120 and the second ultraviolet light source 1130 are actuated (powered on).

In some embodiments, the sterilizing system 1000 can use both ultraviolet light and ozone for sterilizing the commence media placed inside the inner container 1100. In other embodiments, the sterilizing system 1000 uses only ultraviolet light for sterilizing the commence media placed inside the inner container 1100.

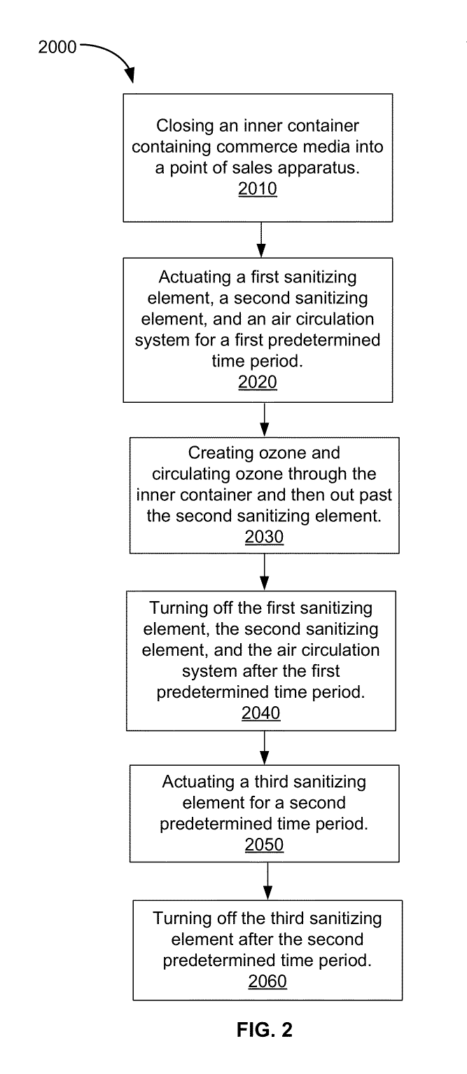

FIG. 2 is an example of a flow chart showing a specific method to destroy pathogenic agents on multiple conveyances of monetary exchange via the use of the commerce media sterilizing system, according to an embodiment. In some embodiments, both UV light and ozone are used as germicidal agents.

The method 2000 includes moving an inner container containing commerce media into, for example, a point of sale apparatus, at 2010. The point of sale apparatus can be, for example, an electronic cash register system. The inner container can be for example, a currency drawer, a cash drawer, a coin drawer, a check drawer, a food stamp drawer, and/or the like. The commerce media can be, for example, paper currency, coins, checks, gift certificates, food stamps, and/or the like.

The method 2000 includes actuating a first sterilizing element, a second sterilizing element, and an air circulation system for a first predetermined time period, at 2020. In step 2020, actuating the first sterilizing element, the second sterilizing element and the air circulation system involves turning on or powering the sterilizing elements and the air circulation system for a first predetermined time period. In some embodiments, the first sterilizing element, at 2020, can be a Vacuum Ultraviolet (VUV) light source(s) configured to create ozone. VUV light can create ozone because the shorter (100 to 200 nm range) wavelength of VUV light reacts with atmospheric oxygen (O₂) to break it into atomic oxygen (O). Atomic oxygen (O) however, is a highly unstable atom that can readily combine with oxygen (O₂) to form ozone gas (O₃). Ozone gas can kill pathogens such as bacteria, viruses, microorganisms, molds, fungus and so forth that exist on the surface of commerce media by rupturing the cell wall of the pathogen and can provide effective germicidal benefits by complementing the germicidal benefits obtained via direct UV light radiation.

In some embodiments, the first sterilizing element can also be, for example, an ultraviolet light source such as an ultraviolet-C (UV-C 200 to 280 nm) light source, also known as germicidal UV. Additionally, in some embodiments, actuating the first sterilizing element can include, for example, turning on or powering the sterilizing element for a first predetermined time period.

The method 2000 includes creating ozone as described above and circulating ozone through the inner container and then out past the second sterilizing element, at 2030. Creating ozone for circulation over and around the enclosed contents (commerce media) of the inner container can further increase the germicidal beneficial effects on commerce media. For the embodiment discussed in method 2000, the second sterilizing element can be an ozone destroying UV light source (such as, for example, a UV-C or a UV-B light source) which can convert ozone back to oxygen and can be located at, for example, near the venting perforations of the outer casing (as described in detail below). Circulating ozone past the second sterilizing element ensures any remaining ozone is converted back to oxygen. The remaining ozone gas can be, for example, the leftover ozone that was not involved in the sanitization of the organic pathogens on the commerce media. The air circulation system can include for example, an exhaust fan in fluid communication with perforations in the outer casing that can allow for air inflow and air outflow from the container.

In some embodiments, the air circulation system can allow for circulating the ozone in and around the commerce media, thereby providing effective sterilization. In some embodiments, the inner container can be, for example, a modified currency drawer which the paper and coin currency can be placed. Such a modified currency drawer can have venting perforations in the body to allow the ozone to be drawn down and through the multiple layers of commerce media. The duration of the first predetermined time period is set to be sufficient to draw all of the ozone though the currency drawer by way of the ventilation perforations, past the ozone destroying UV light source (200-300 nm wavelength range), where the ozone is converted to oxygen, and then expelled out of the outer casing.

The method 2000 includes turning off the first sterilizing element, the second sterilizing element, and the air circulation system after the first predetermined time period, at 2040. As such, all ozone produced is converted to oxygen and expelled from the body of the container in a sufficient time such that there is no danger of exposing a user of the apparatus to potentially harmful ozone when the inner container moves to its open configuration, for example.

The method 2000 includes actuating a third sterilizing element for a second predetermined time period, at 2050. In step 2050, actuating the third sterilizing element involves, turning on or powering the sterilizing element for a second predetermined time period, whereby the second predetermined time period is either the same amount of time or a different amount of time from the first predetermined time period. In step 2050, the third sterilizing element can be a UV-C light source(s) that can be located, for example, directly above the currency drawer. The UV-C light source(s) is turned on or powered on in step 2050 for the germicidal treatment of the currency media. The UV-C radiation can kill various kinds of pathogens such as for example, bacteria, virus, microorganisms, molds, fungus and so forth that exist on the surface of commerce media with direct light illumination. The UV radiation can kill the different kinds of pathogens by damaging the DNA. UV radiation disrupts the chemical bonds that hold the atoms of DNA together in the pathogen. If the damage is severe enough, the pathogen cannot repair the damage and will die. For the case of paper currencies, the UV-C light can also penetrate through the upper bills allowing a diminishing portion of the UV-C light to effect the underlying bills.

The method 2000 includes turning off the third sterilizing element after the second predetermined time period, at 2060. By the end of step 2060, the inner container is safely moved to its open configuration.

If, at anytime during any of the steps of method 2000, the user opens the inner container from the outer casing, power from the power supply to the sterilizing elements is turned off, and/or the air circulation system is turned off, and/or all internal electrical cycles are interrupted. This ensures the whole sterilizing system is turned off.

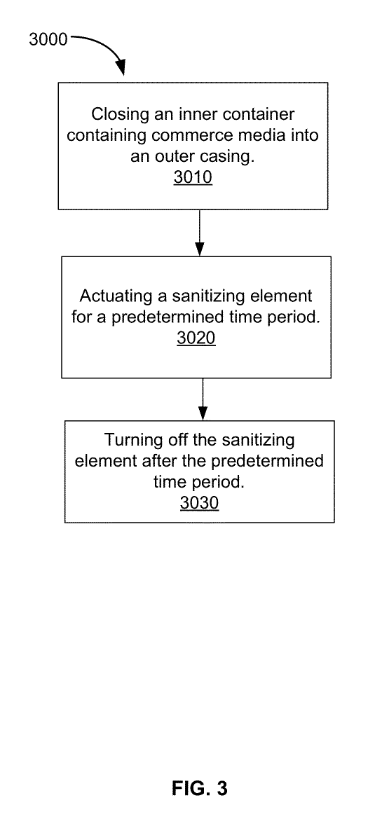

FIG. 3 is an example of a flow chart showing a method to destroy pathogenic agents on multiple conveyances of monetary exchange via the use of the commerce media sterilizing system, according to an embodiment. In some embodiments only UV light (200 to 280 nm wavelength) is used as the germicidal agent.

The method 3000 includes moving an inner container containing commerce media from an open configuration to a closed configuration such that the inner container is closed within an outer casing, at 3010. Similar to step 2010 in method 2000 (FIG. 2) described above, the outer casing can be, for example, a point of sale apparatus such as an electronic cash register system. The inner container can be for example, currency drawer, a cash drawer, a coin drawer, a check drawer, a food stamp drawer, and/or the like. The commerce media can be, for example, paper currency, coins, checks, gift certificates, food stamps, and/or the like.

The method 3000 includes actuating a sterilizing element for a predetermined time period, at 3020. In some embodiments, the sterilizing element is a UV-C light source. In step 3020, actuating the sterilizing element includes turning on or powering the UV-C light source for a pre-determined time period. As described above in method 2000, the UV light can act as a germicidal agent by killing bacteria and other pathogenic agents on commerce media.

The method 3000 includes turning off a sterilizing element after a predetermined time period, at 3030. A design parameter for such a germicidal system includes determining the appropriate pre-determined time period to keep the sterilizing element turned on. Longer exposure times to UV light can generally ensure increased (or maximum) destruction of all the pathogens on the commerce media. For POS apparatus such as electronic cash register systems, the time period the drawer can be closed inside the inner chamber can vary depending on the type of business, peak hour business rate, time of the year, special holiday sales events, and or the like.

Note that the embodiment described in FIG. 3 need not use ozone as a germicidal agent. Hence there is no need for an air circulation system to pass the ozone through the inner container of the point of sale apparatus that can contain different layers of commerce media, draw out the remaining (unreacted) ozone gas, convert the ozone gas to oxygen and expel it from the inner container and the outer casing of the POS apparatus. Additionally, in some embodiments, only one UV light source is used. Hence, such an embodiment can be less complex and more cost-effective.

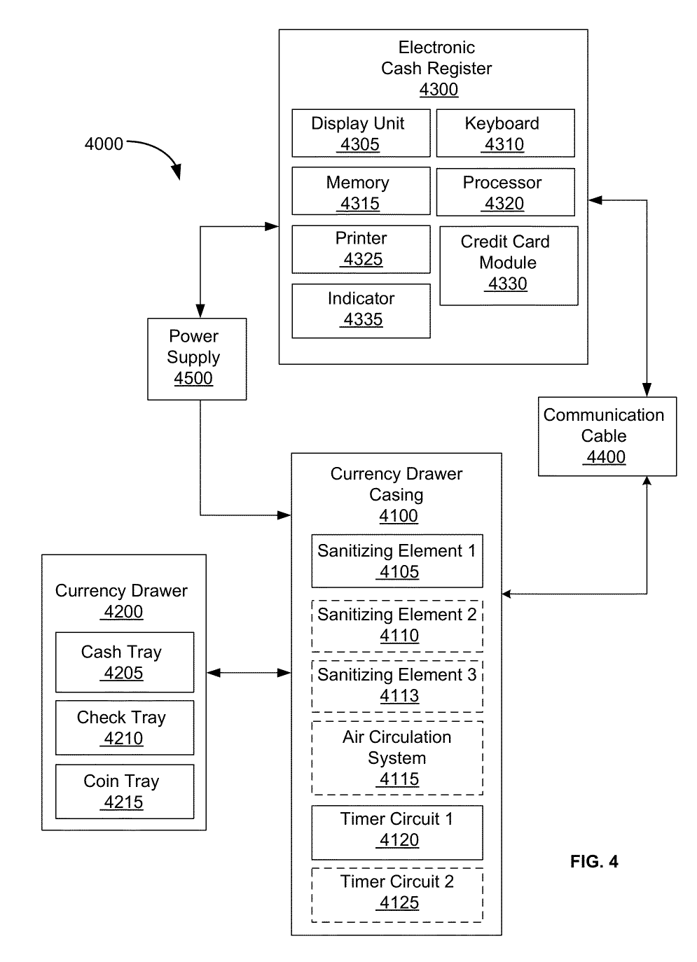

In some embodiments, the methods shown and described herein can be performed using a point of sale apparatus of the type shown and described in FIGS. 4-10. FIG. 4 is a block diagram of a point of sale apparatus that includes a commerce media storage and sterilization system, according to an embodiment. The apparatus 4000 include a currency drawer casing 4100, a currency drawer 4200, an electronic cash register 4300, a communication cable 4400, and a power supply 4500.

The currency drawer casing 4100 can be made of a number of materials such as, for example, steel, Acrylonitrile Butadiene Systrene (ABS) plastic, and/or so forth. The currency drawer casing 4100 can substantially enclose an interior volume that can be configured to receive a currency drawer 4200 and can also tightly seal the currency drawer 4200 within. Additionally, the currency drawer casing 4100 can also include a first sterilizing element 4105, and optionally a second sterilizing element 4110 (as indicated by the dashed lines), a third sterilizing element 4113 (as indicated by the dashed lines), a first timer circuit 4120, optionally a second timer circuit 4125 (as indicated by the dashed lines), and optionally an air circulation system 4115 (as indicated by the dashed lines).

In some embodiments, when only the UV-C light source is used as the germicidal agent, the first sterilizing element 4105 can be a UV-C light source. The germicidal wavelength (200-280 nm) radiation of the UV-C light can directly destroy the genetic material of germs. In such embodiments, while the second sterilizing element 4110, or the third sterilizing element 4113 are not required, such elements can be provided.

In some embodiments, when both UV light and ozone are used as germicidal agents, the first sterilizing element 4105 can be a VUV light source. The shorter wavelengths of radiation between 100-200 nm (higher energy radiation) associated with the VUV light have the capability to create ozone from atmospheric oxygen. In such embodiments, prior to the ozone being exhausted from the currency drawer casing 4100, a second sterilizing element 4110 is used to convert the ozone back to oxygen before being expelled from the currency drawer casing 4100. In some embodiments, the second sterilizing element 4110 can be ozone destroying UV-C or UV-B light source(s) that emit radiation in the range of 200-300 nm. The longer wavelength (lower energy) radiation of the UV-C or UV-B light can be used to convert any unreacted ozone gas back to atmospheric oxygen before being expelled from the currency drawer casing 4100. In some embodiments, a third sterilizing element can be employed to directly use UV light illumination to kill pathogenic agents on currency media. The third sanitizing element 4113 can be germicidal UV-C light source(s). The germicidal wavelength (200-280 nm) radiation of the UV-C light can directly destroy the genetic material of germs.

The air circulation system 4115 can include, for example, an exhaust fan with associated perforations in the currency drawer casing 4100 and other ventilation perforations that can allow for air inflow and air outflow. In some embodiments, when only the UV-C light source is used as the germicidal agent, an air circulation system 4115 may or may not be utilized. The lack of ozone gas diminishes the need for an air circulation system 4115. In other embodiments, where both UV light and ozone are used as the germicidal agents, the air circulation system 4115 can be used to pass the ozone through the inner volume of the currency drawer casing 4100 that can contain the currency drawer 4200 with the commerce media, draw out the remaining (unreacted) ozone gas, convert the ozone gas to oxygen and expel it from the inner volume of the currency drawer casing 4100.

The timer circuits 4120 and 4125 are configured to control the actuation of the sterilizing element(s) 4105 and/or 4110 and/or 4113, and/or the air circulation system 4115. In some embodiments, the timer circuits 4120 and 4125 can be activated by a signal transmitted by, for example, a relay switch that can indicate when the currency drawer 4200 is disposed inside the casing 4100 and is in a closed and/or sealed position. Upon activation, the timer circuits 4120 and/or 4125 allow the conduction of power from the power supply 4500 to the sterilizing element(s) 4105 and/or 4110 and/or 4113 and the air circulation system 4115 for a predetermined time period. This can activate the sterilizing element(s) 4105 and/or 4110 and/or 4113 and the air circulation system 4115 for a predetermined time period.

In some embodiments, when only UV light is used as the germicidal agent, timer circuit 4120 can actuate sterilizing element 4105 (which can be a UV-C light source) for a predetermined period of time sufficient for the destruction of pathogenic organic matter on the commerce media, thus rendering the commerce media sterilized/sanitized. In such embodiments, timer circuit 4120 can also deactivate the sterilizing element 4105 at the conclusion of the predetermined time period. In some embodiments, the timer circuit 4120 is interrupted in the event the currency drawer 4200 is opened (outside the currency drawer casing 3100) during the active period of the timer circuit 4120.

In some embodiments, when both UV light and ozone gas are required as the germicidal agents, both timer circuits 4120 and 4125 can be used to implement the commerce media sterilization methods. In such embodiments, timer circuit 4120 can actuate sterilizing element 4105 (which can be a VUV light source), sterilizing element 4110 (which can be an ozone destroying UV-B or UV-C light source) and the air circulation system 4115 for a first predetermined period of time. In such embodiments, timer circuit 4120 can also deactivate the sterilizing element 4105, sterilizing element 4110, and the air circulation system 4115 at the conclusion of the first predetermined time period, or if the timer circuit 4120 is interrupted in the event the currency drawer 4200 is opened (outside the currency drawer casing 4100) within the timer circuit 4120 active period. The time period of activation for timer circuit 4120 should be set to a value that is sufficient to draw all ozone gas though the currency drawer 4200 by way of, for example, the ventilation perforations, past an ozone destroying light source such as 4110 (UV-B or UV-C light source), where the ozone is converted to oxygen which can then be exhausted out of the body of the currency drawer casing 4100.

In such embodiments, timer circuit 4125 can also actuate sterilizing element 4113 (which is a UV-C light source) for a second predetermined period of time for the direct UV light based germicidal treatment of pathogens on the currency media. The timer circuit 4125 can also deactivate the sterilizing element 4113 at the conclusion of the second predetermined time period, or if the timer circuit 4125 is interrupted in the event the currency drawer 4200 is opened within the timer circuit 4125 active period.

The currency drawer 4200 can be made any kind of material, such as, for example, steel and/or so forth. The currency drawer 4200 can include one or more levels of cash trays 4205, check trays 4210, and/or coin trays 4215. The cash tray(s) 4205 can hold any kind of cash or other kinds of paper currency. The check tray(s) 4210 can hold personal checks, traveler's checks, money orders, gift certificates, foot stamps, and/or so forth. The coin tray(s) 4215 can hold coins and any other form of metal based currency. In some embodiments, the currency drawer 4200 can also have ventilation perforation plates (not shown in FIG. 4) around one or more of the cash trays(s) 4205, check tray(s) 4210, and coin tray(s) 4215. Such ventilation perforation plates can include an ordered array of openings that can help create useful air flow channels around the commerce media thus allowing efficient passage of ozone for increased sterilization of the commerce media contained inside the currency drawer 4200.

The electronic cash register 4300 is an electronic device for calculating and recording sales transactions that can include an attached currency drawer 4200 for storing cash and other forms of commerce media. The electronic cash register 4300 can include a display unit 4305, a keyboard 4310, a memory 4315, a processor 4320, a printer 4325, a credit card module 4330, and an indicator 4335. However, the electronic cash register 4300 and the currency drawer 4200 can be separate components of a POS apparatus that can be linked together by the power supply 4500 and the communication cable 4400, or in any other configuration. In some embodiments, the apparatus can be an automatic teller machine.

The display unit 4305 can be, for example, a liquid crystal display (LCD) unit or a light emitting diode (LED) alpha-numeric display unit. In some embodiments, there can be only one display unit 4305 for the cashier. In other embodiments, there can be two display units 4305, one for the cashier and a second rotatable display unit for the customer. The display unit 4305 can also include additional features such as, for example, the price lookup function (PLU) that can allow the cashier to look up the prices of a predetermined number of items.

The keyboard 4310 can be any standard electronic cash register keyboard that can have any number of keys that can be, for example, raised keys, flat keys, spill proof keys, and so forth. In addition to the standard keyboard keys such as the numeric keys, the keyboard 4310 can have additional customized keys such as, for example, sales tax key, currency converter key, remote printer send key (for printing a paper receipt at a remote printer), bill separation key, birthday entry key, and/or so forth.

The memory 4315 can be, for example, a random access memory (RAM), a memory buffer, a hard drive, a database, an erasable programmable read-only memory (EPROM), an electrically erasable read-only memory (EEPROM), and/or so forth. The memory 4315 can include a database that can contain entries associated with the price of different items in a commercial enterprise corresponding to an identifier for the item. Additionally, the memory can also store information that can be entered by the cashier such as new sales tax rates, discounts on items included in a sale, currency converter rates, change in price of items, and/or so forth. The memory 4315 can also include a dedicated data base that can have entries that can be accessed by the processor 4320 and which can contain instructions for the processor 4320 to execute different functions such as, for example, add, multiply, compute tax on a purchase, determine taxable purchases, split up a bill, void a transaction, and/or the like.

The processor 4320 can be, for example, a general purpose processor, a Field Programmable Gate Array (FPGA), an Application Specific Integrated Circuit (ASIC), a Digital Signal Processor (DSP), and/or the like. The processor 4320 is operatively coupled with the memory 4315 and can be configured to run and/or execute processes, and/or other modules, and/or functions associated with the point of sale apparatus 4000.

The printer 4325 can used to print receipts of purchase for customers of a commercial enterprise and can be either directly integrated into the electronic cash register 4300 or remotely located that can communicate to the electronic cash register 4300 via a wireless or wired communication link. The printer 4325 can be a thermal printer, a drum-based inkjet printer, a laser printer, and/or so forth. In some embodiments, the printer 4325 can have logo printing capability that can allow printing of coupons or advertising messages for a commercial enterprise.

The credit card module 4330 is a module that can be used for credit card and debit card purchases and/or transactions. The credit card module 4330 can be either directly integrated into the electronic cash register 4300 or remotely located that can communicate to the electronic cash register 4300 via a wireless or wired communication link. The credit card module 4330 can include, for example, a credit card reader, a memory, and a database associated exclusively with credit card purchases and/or transactions. In some embodiments, the dedicated memory and processor can be a part on the memory 4315 and processor 4320 of the electronic cash register 4300. In other embodiments, the dedicated memory and processor can be separate hardware modules associated only with the credit card module 4330.

The indicator 4335 can be an LCD or LED display that can be associated with the sterilization of the commerce media in the currency drawer 4200 while it is inside the currency drawer casing 4100. For example, while the UV light and/or ozone treatment of the commerce media is ongoing, the indicator 4335 can turn on (light up), which can inform the cashier (or user) that the sterilization methods are proceeding. After the UV light and/or ozone treatment is completed, the indicator 4335 can turn off which can inform the cashier (or user) that it is safe to open the currency drawer 4200 from the currency drawer casing 4100.

The communication cable 4400 can transmit various kinds of electrical signals from the currency drawer casing 4100 to the electronic cash register 4300 and vice-versa. In some embodiments, the communication cable 4400 can be a standard RS232 cable, a parallel port connection cable, a universal serial bus (USB) cable, and /or so forth. In other embodiments, communication is achieved wirelessly. In some embodiments, electrical signals can be sent from the currency drawer casing 4100 to the electronic cash register 4300 when a drawer catch/release mechanism in the currency drawer casing 4100 (not shown in FIG. 4) is activated upon closure (and/or locking) of the currency drawer 4200 within the currency drawer casing 4100. This signal can inform the electronic cash register 4300 about the status of the currency drawer 4200. In other embodiments, when the cashier (or user) needs to open the currency drawer 4200, a specific key in the keyboard 4310 of the electronic cash register 4300 can be pressed, for example, a “cash” button, and/or so forth. Pressing the specific key can send an electrical signal from the electronic cash register 4300 via the communication cable 4400 to the currency drawer casing 4100 that can release the drawer catch/release mechanism. This can release the currency drawer 4200 from the locked position inside the currency drawer casing 4100 allowing the currency drawer 4200 to open.

The power supply 4500 can include a standard direct (DC) power supply cord with an alternate current (AC) adapter that can be plugged into any standard wall power outlets. In some embodiments, the power supply 4500 can involve only one standard power supply cord connecting the electronic cash register 4300 to a wall power outlet. In such embodiments, a second power cord is used to supply power from the electronic cash register 4300 to the currency drawer casing. In other embodiments, two standard direct (DC) power supply cords can be employed, whereby the first power cord can connect the electronic cash register 4300 to the power wall outlet, and the second power cord can connect the currency drawer casing 4100 to the power wall outlet.

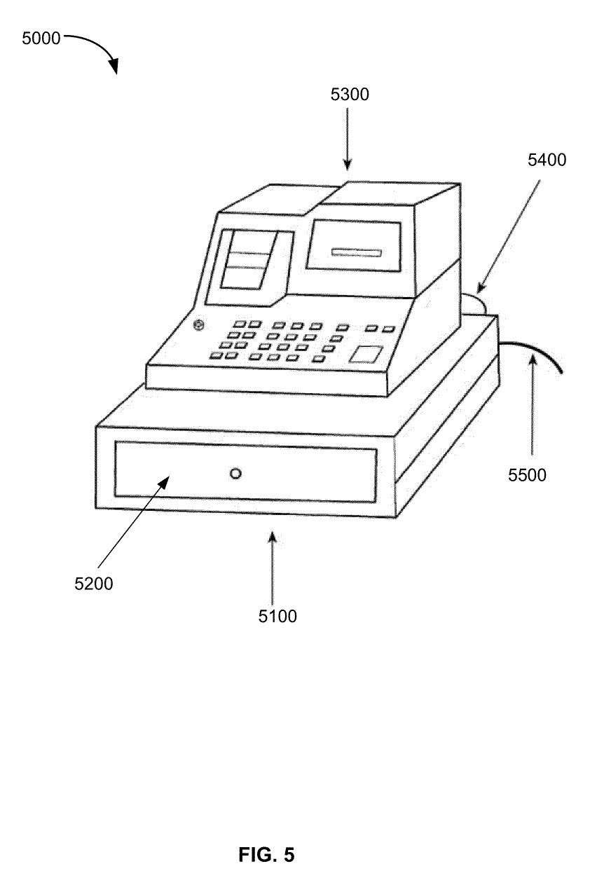

FIG. 5 is a point of sale apparatus, according to an embodiment, which is configured to implement the commerce media sterilizer according to the invention. The point of sale apparatus 5000 includes a currency drawer casing 5100, a currency drawer 5200 that can be closed inside the currency drawer casing 5100, an electronic cash register 5300, a communication cable 5400, and a power supply (cord) 5500.

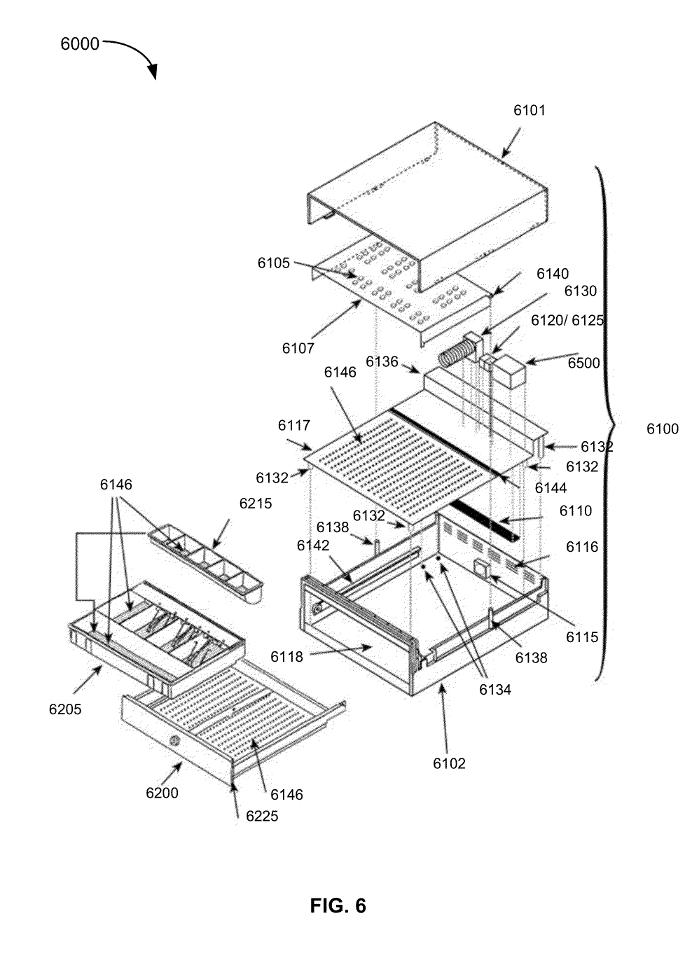

FIG. 6 is an exploded perspective view of a currency storage and sterilization module of a point of sale apparatus that is configured to implement a method of sterilizing commerce media via the use of UV light and ozone, according to an embodiment.

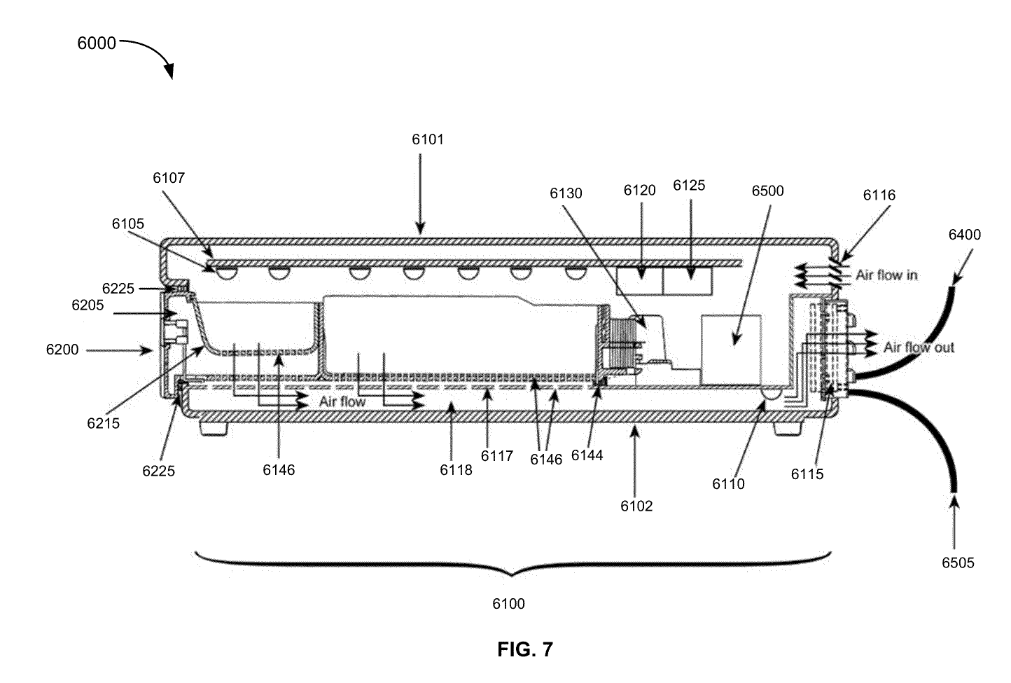

FIG. 7 is an enlarged cross-sectional view of a currency storage and sterilization module of a point of sale apparatus that can implement a method of sterilizing commerce media via the use of UV light and ozone, according to an embodiment.

The description of components of the currency storage and sterilization module 6000, the relationship of the components with one another, and their function in some embodiments are provided with reference to FIGS. 6 and 7.

The currency storage and sterilization module 6000 can include a currency drawer casing 6100, a currency drawer 6200, a communication cable 6400, and a power supply 6500. The currency drawer casing 6100 includes a cover casing 6101, a body casing 6102, UV light source(s) 6105, a UV light source mounting plate 6107, UV light source(s) 6110, an exhaust fan 6115, air inlet vents 6116, an air flow plate 6117, an airflow chamber 6118, timer a 6120, timer b 6125, a drawer catch/release mechanism 6130, screw posts 6132, mounting points 6134, a flexion region 6136, bosses 6138, plate projects 6140, guide rails 6142, a rear currency drawer gasket 6144, and ventilation perforations 6146.

The currency drawer 6200 includes a cash tray 6205, a coin tray 6215, a forward currency drawer gasket 6225, and ventilation perforations 6146 on several surfaces. The communication cable 6400 can transmit electrical signal pulses which can control a drawer catch/release mechanism 6130 within the currency drawer casing 6100. This electrical signal can cause the drawer catch/release mechanism 6130 to release the currency drawer 6200 from a locked position allowing it to open laterally and forward on guide rails 6142 for access to a cashier or user.

The power cord 6505 can supply electricity to electrical devices contained within the currency storage and sterilization module 6000. Power may be derived, for operation of the currency storage and sterilization module 6000 from, for example, a direct power source such as power wall outlet or a electric generator, or a direct power source in tandem with power supplied by the electronic cash register 5300 (in FIG. 5).

In some embodiments, the UV light source mounting plate 6107 can be of equal width and length with that of the currency drawer 6200 such that uniform exposure of all commerce media in the currency drawer 6200 to the UV light can occur for sterilization. Attached to the underside of the UV light source mounting plate 6107 can be an array of UV light source(s) 6105 that can be evenly distributed to maximize UV light exposure of the commerce media. The UV-light sources can be, for example, VUV light sources, and/or UV-C light sources. In some instances, the UV light source(s) 6105 can be VUV light sources when the generation of ozone is required. In other instances, the UV light source(s) 6105 can also be UV-C light sources when the germicidal properties of direct UV light illumination need to be harnessed.

In some embodiments, the UV light source mounting plate 6107 can be mounted on the cover casing 6101 such that the UV light source(s) 6105 are positioned directly above the recessed currency drawer 6200. This configuration can allow the UV light to directly impinge upon the surface of the commerce media contained within the cash tray 6205 and coin tray 6215 when the UV light source(s) are powered on. However, in other embodiments, the UV light source mounting plate 6107 can be mounted in any other configuration with respect to the recessed currency drawer 6200.

The mounting of the UV light source mounting plate 6107 can be made to the cover casing 6101 or, as in the example presented herein, to the body casing 6102 by securing with plate projects 6140 and bosses 6138 or any other securing devices that can maintain the UV light source mounting plate 6107 in appropriate relationship to the currency drawer 6200.

The air flow plate 6117 with ventilation perforations 6146 can create an air flow chamber 6118 when joined to the body casing 6102 with screw posts 6132 affixing to mounting points 6134 as illustrated in FIG. 6. A recess 6136 in the air flow plate 6117 can be utilized in this embodiment to accommodate the vertical placement of an exhaust fan 6115. The ventilation perforations 6146 need to be created and placed specifically to maximize air flow between individual units of commerce media in order to assure maximum exposure of the commerce media to the ozone. The purpose of air circulation is to utilize the germicidal properties of ozone that can be produced by the ozone producing UV light source(s) 6105 (VUV light source), by drawing the ozone down over the cash and coin currency, thus further sterilizing the commerce media to a degree that direct UV light exposure may not.

In some embodiments, a mechanism for limiting movement or vibration of UV light sources 6105 during opening and closing of the currency drawer 6200 into (and out of) the currency drawer casing 6100 and during vibrations caused by air circulation and exhaust fan 6115 can be provided. This can allow the UV light source(s) 6105 to be securely held in the mounting plate 6107 thereby reducing any risk of any of the UV light source(s) 6105 from breaking off the mounting 6107 and crashing on to the currency drawer 6100.

For example, the UV-light source mounting plate 6107 can include a safety lock mechanism (not shown in FIGS. 6 and 7) in one or multiple corners. The safety lock mechanism can include a stopper and an actuator. The stopper of the safety lock mechanism can be a protrusion extending from the proximal surface of the safety lock mechanism. The stopper can be configured to change positions either via applying manual force or via a mechanical force applied by the actuator. The stopper can be configured to make contact with a portion of the UV-light source mounting plate 6107 once the UV-light source mounting plate 6107 is mounted on the currency drawer casing 6100. This can securely hold the UV-light source mounting plate 6107 (housing the UV light source(s) 6105) on the currency drawer casing 6100 and can prevent or limit the lateral movement of the UV light source(s) 6105 during vibrations caused by air circulation and the exhaust fan and during vibrations caused by the closing of the currency drawer 6200 into (and out of) the currency drawer casing 6100.

In some embodiments, the actuation device of the safety lock mechanism can include a mechanical energy storage member, such as a spring, a device containing compressed gas, a device containing a vapor pressure-based propellant or the like. In other embodiments, the actuation device of the safety lock mechanism can include an electrical energy storage member, such as a battery, a capacitor, a magnetic energy storage member or the like. In yet other embodiments, the actuation device can include an energy storage member can be a chemical energy storage member, such as a container containing two substances that, when mixed, react to produce energy (i.e., a propellant).

Once the currency drawer 6200 is moved to a closed position by a cashier or user sliding it laterally along guide rails 6142, the currency drawer 6200 can engage with the drawer catch/release mechanism 6130.

In some embodiments, the drawer catch/release mechanism 6130 can contain and/or utilize a relay switch in one form or another that can be either a part of the currency drawer 6200, the currency drawer casing 6100, or can be associated with the currency drawer 6200 in any other configuration. The relay switch can send electrical signals to the electronic cash register 5300 (in FIG. 5) that can indicate the closed and/or locked configuration of the currency drawer 6200 with respect to the electronic cash register 5300. Once the drawer catch/release mechanism 6130 is engaged with a closed currency drawer 6200, the relay switch device is activated and can activate timers 6120 and 6125 that can begin the process of conducting power for a predetermined period to the UV light source(s) 6105 and 6110 and the exhaust fan 6115.

In other embodiments, the existing currency drawer 6200/currency drawer casing 6100 design may not be suitable to implement relay switches. In such embodiments, the drawer catch/release mechanism 6130 can utilize, for example, a switch biased to an off position to activate the timers 6120 and 6125. In such embodiments, the currency drawer 6200 comes in contact with the switch when the currency drawer 6200 is in a closed position inside the currency drawer casing 6100 (the currency drawer 6200 may or may not be locked inside the currency drawer casing 6100). This can, for example, turn the switch from an “off” to an “on” position and thus can activate the timers 6120 and 6125.

In some embodiments discussed herein (e.g., an apparatus including a relay switch and/or a non-relay switch), once the switch is activated, timer 6120 can facilitate the conduction of power from power supply 6500 to activate the UV light source(s) 6105 for a first predetermined period that is sufficient for the destruction of surface born organic matter. In the embodiment described in FIGS. 6 and 7, the UV light source(s) 6105 can be VUV and/or UV-C light sources. When employing a VUV light source 6105, ozone can be created that has a germicidal effect on the target commerce media when the ozone is drawn down, over the commerce media and through the currency drawer 6200. Thus, VUV light illumination can render the target commerce media sterilized.

Timer 6120 can deactivate the UV light source(s) 6105 at the conclusion of the first predetermined period, or if interrupted when the drawer catch/release mechanism 6130 releases the currency drawer 6200 to an open position during the timer 6120 active period. In some embodiments, the drawer catch/release mechanism 6130 can receive an electrical impulse/signal from the electronic cash register 6300 to unlock the currency drawer 6200. This can allow the currency drawer 6200 to move to an open position.

In other embodiments, timer 6120 can conduct power to activate, substantially in unison, the exhaust fan 6115, the ozone producing UV light source(s) 6105 (e.g., VUV light sources), and the ozone destroying light source(s) 6110 (which can be either UV-C or UV-B light sources) for a first predetermined time period. The air circulation begins on activation of the exhaust fan 6115, thus causing the draw of air in though air inlet vents 6116 and past the UV light source(s) 6105, where ozone is created. The newly created ozone can be drawn down through the currency drawer 6200 and over the many surfaces of the commerce media residing within, by the ventilation perforations 6146 that are part of the coin tray 6215, cash tray 6205, currency drawer 6200, and the airflow plate 6117. The ozone can then be drawn through the airflow chamber 6118, past the ozone destroying UV light source 6110, which can convert the ozone back into oxygen. The oxygen can then be harmlessly vented past exhaust fan 6115 and out of the back of the currency drawer casing 6100. Hence, the duration of activation of timer 6120 needs to be sufficiently long to allow the above mentioned functions to be performed properly.

In some embodiments, to assure air flow integrity, a sealing arrangement can be utilized to prevent undesirable air flow paths. For example, when the currency drawer 6200 is recessed and locked within the body casing 6102 it can come in contact with a rear cash drawer gasket 6144 and a forward cash drawer gasket 6225 which can form a mechanical seal between the currency drawer 6200 and the body casing 6102. This can prevent leakage of air from or into the currency drawer 6200 and/or the currency drawer casing 6100. In other embodiments, other gaskets and additional/alternate placements may be utilized to assure air flow integrity.

Timer 6120 can deactivate, substantially in unison, the exhaust fan 6115, the ozone producing UV light source(s) 6105 (VUV light sources), and the ozone destroying light source(s) 6110 (which can be either UV-C or UV-B light sources) at the conclusion of the first predetermined time period. Full completion of the activation period of timer 6120 is important to assure all remaining ozone gas is drawn out and converted to oxygen and exhausted from the body casing 6102.

In some embodiments, timer 6125 can also conduct power to activate the germicidal UV light source(s) 6105 (UV-C light sources) for a second predetermined time period. This can cause direct UV illumination to impinge upon the commerce media placed in the cash tray 6205. This step can add additional germicidal benefits for the sterilization of the commerce media. For the case of paper currency, direct illumination of the UV-C light can also penetrate through the upper bills allowing a diminishing portion of the UV-C light to effect the underlying bills. Timer 6125 can also deactivate the germicidal UV light source(s) 6105 at the conclusion of the second predetermined time period. Both the timers 6120 and 6125 cycle once per closing of the currency drawer 6200 inside the currency drawer casing 6100 in the embodiments described in FIGS. 6 and 7 whereby both ozone and direct UV light are used as the germicidal agents.

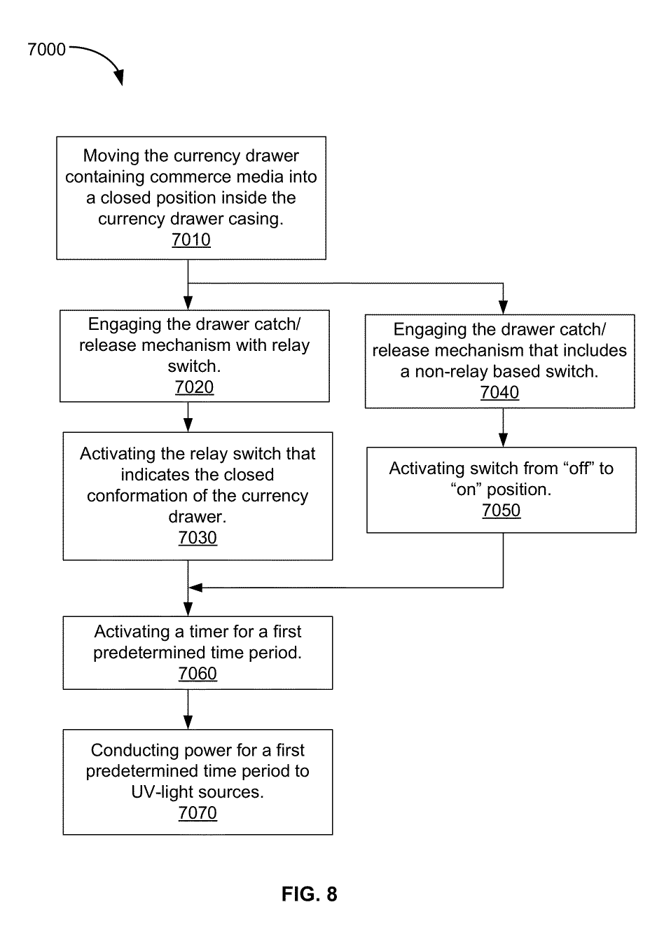

FIG. 8 is an example of a flow chart showing methods of activating UV-light sources, according to different embodiments. The method 7000 includes moving the currency drawer containing commerce media into a closed position inside the currency drawer casing, at 7010. Once the currency drawer is in a fully closed position inside the currency drawer casing, it may or may not be locked and/or sealed.

The method 7000 includes engaging the drawer catch/release mechanism with a relay switch, at 7020. The drawer catch/release mechanism can include, for example a coiled compression spring and a transducer. The coiled compression spring can be compressed to its shortest length when the currency drawer is pushed all the way inside the currency drawer casing. This can activate a transducer that is associated with the coiled compression spring.

The method 7000 includes activating the relay switch that indicates the closed condition of the currency drawer to the electronic cash register, at 7030. Activation of the transducer of the drawer catch/release mechanism can send an electrical signal to activate the relay switch. As described above, the relay switch can be a part of the currency drawer, the currency drawer casing, or can be associated with the currency drawer in any other configuration. Once activated, the relay switch can send electrical signals to the electronic cash register that can indicate the closed configuration of the currency drawer to the electronic cash register.

Alternatively, in some embodiments where the existing currency drawer/currency drawer casing design may not be suitable to implement relay switches, the method 7000 can include engaging the drawer catch/release mechanism that can include a non-relay based switch, at 7040. The switch can be, for example, initially in an “off” configuration when the currency drawer is outside the currency drawer casing.

Alternatively, in such embodiments, the method 7000 can include activating the switch from an “off” to an “on” position, at 7050. Once the currency drawer is in a fully closed position inside the currency drawer casing, the currency drawer can come in contact with the switch and change its configuration from the “off” position to an “on” position. This can activate the switch.

After the switch is activated, the method 7000 includes activating a timer for a first predetermined time period, at 7060.

The method 7000 further includes conducting power for a first predetermined time period to UV-light sources, at 7070. As described above, the activated timer can allow power to be conducted from a power supply unit to activate one or more UV light source(s) for a first predetermined time period. The first predetermined time period is set to be long enough to ensure the destruction of surface born pathogenic matter such as, for example, bacteria, viruses, molds, fungi, and/or so forth. The UV light illumination process can render the target commerce media sterilized.

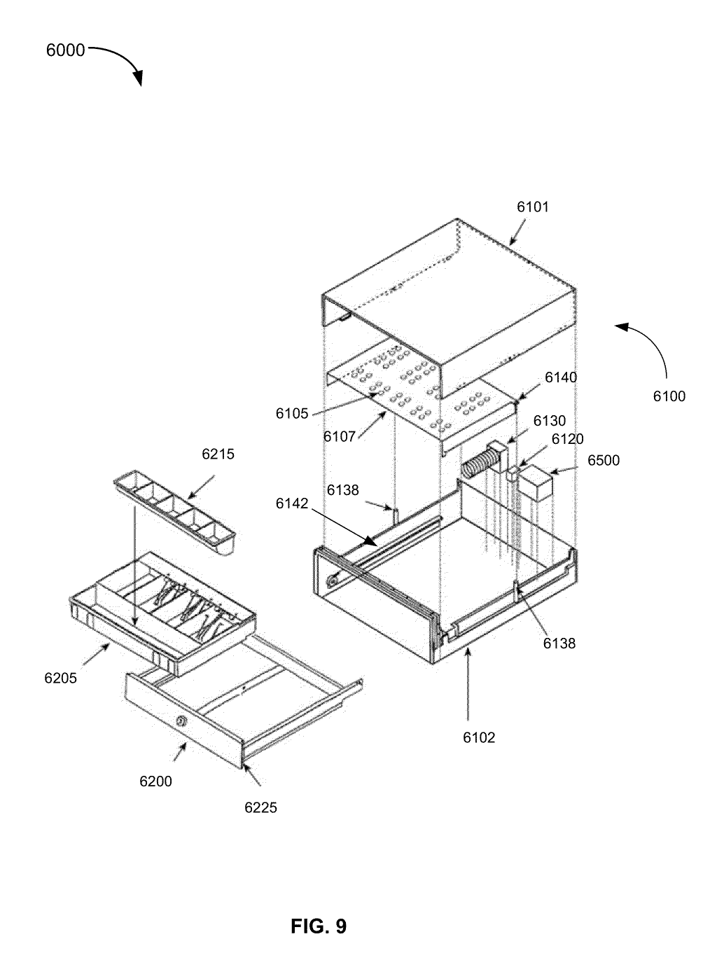

FIG. 9 is an exploded perspective view of a currency storage and sterilization module of an apparatus that configured to sterilize commerce media via the use of UV light only, according to an embodiment.

In FIG. 9, the germicidal UV-light source(s) 6105 can be, for example, UV-C light source(s) since UV light is the only germicidal agent employed in this embodiment. In the embodiment illustrated in FIG. 9, all components numbered in common with FIGS. 6 and 7 have similar functionality as described above.

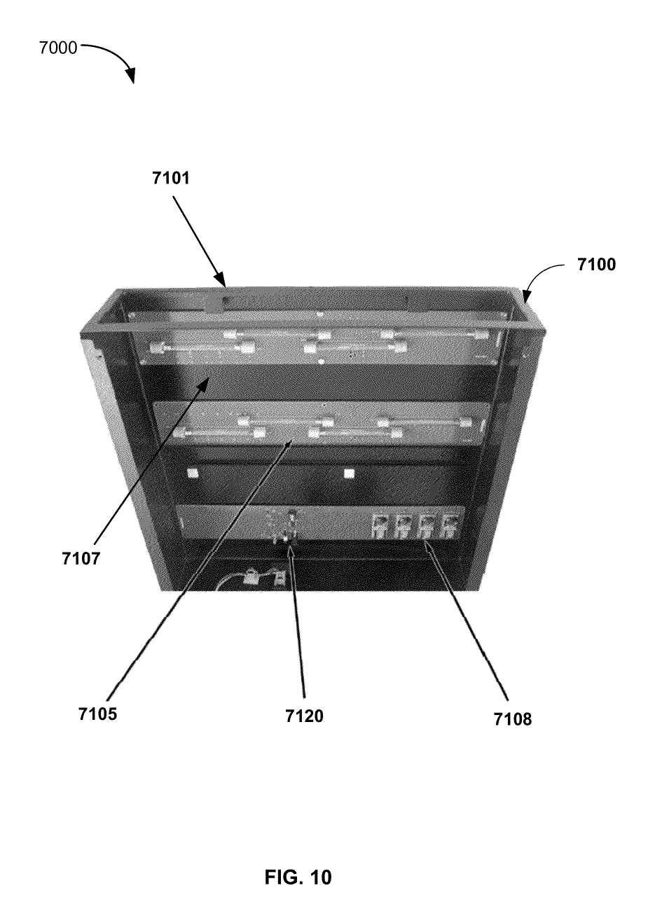

FIG. 10 illustrates an embodiment of a currency sterilization device, according to an embodiment. The device 7000 includes a currency drawer (not shown in FIG. 10) and a currency drawer casing 7100. The currency drawer casing 7100 includes a group of UV light source(s) 7105, UV light on/off timer control units 7120, UV bulb inverters 7108 (that can power the UV light source(s)), UV light source mounting plate 7017, and cover casing 7101. In some embodiments, the components of the UV lighting system were designed to fit in a preexisting currency drawer casing 7100 of an existing apparatus with a specified interior clearance between the currency drawer (not shown in FIG. 10) and the upper surface of the currency drawer casing 7100.

The UV light on/off timer control units 7120 can be adjusted for varying length and can be activated utilizing the currency drawer's preexisting drawer latch/switch. In some embodiments, the UV light sources 7105 are located directly above the commerce media in the currency drawer and can radiate the UV light in the downward direction directly upon the commerce media. In other embodiments, the UV light source(s) 7105 can be oriented in any other conformation with respect to the currency drawer.

Some embodiments described herein relate to processors and are associated with a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

While various embodiments have been described above, it should be understood that they have been presented by way of example only, and not limitation, and as such, various changes in form and/or detail may be made. Any portion of the apparatus and/or methods described herein may be combined in any suitable combination, unless explicitly expressed otherwise. Where methods and/or schematics described above indicate certain events and/or flow patterns occurring in certain order, the ordering of certain events and/or flow patterns may be modified. Additionally certain events may be performed concurrently in parallel processes when possible, as well as performed sequentially.

What is claimed is:

1. An apparatus, comprising:
an outer casing configured to substantially enclose an interior volume;
an inner container configured to receive a paper currency;
an actuator configured to be actuated by a movement of the inner container; and
a sterilizing element disposed within the interior volume and configured to effectively sterilize the paper currency based on actuation of the actuator.

2. The apparatus of claim 1, wherein the sterilizing element includes at least one ultraviolet light source.

3. The apparatus of claim 1, wherein the sterilizing element includes at least one germicidal light source.

4. The apparatus of claim 1, wherein the sterilizing element includes at least one of a vacuum ultraviolet (VUV) light source, an ultraviolet-B (UV-B) light source or an ultraviolet-C (UV-C) light source.

5. The apparatus of claim 1, wherein the sterilizing element is configured to be actuated for a predetermined time period.

6. The apparatus of claim 1, wherein the sterilizing element is configured to emit light at a wavelength of between about 100 nm to 300 nm.

7. The apparatus of claim 1, further comprising an exhaust fan configured to move air from within the interior volume to an area outside the outer casing.

8. The apparatus of claim 1, wherein the inner container is configured to move from a first configuration to second configuration, the inner container configured to engage the actuator in the second configuration.

9. A method, comprising:
actuating, after movement of a container configured to hold paper currency from a first position outside of a casing to a second position inside of the casing, a sterilizing element effective to sterilize the paper currency; and
deactivating the sterilizing element.

10. The method of claim 9, wherein the actuating the sterilizing element includes actuating at least one ultraviolet light source.

11. The method of claim 9, wherein the actuating the sterilizing element includes actuating at least one of a vacuum ultraviolet (VUV) light source, an ultraviolet-B (UV-B) light source or an ultraviolet-C (UV-C) light source.

12. The method of claim 9, further comprising actuating an exhaust element configured to exhaust air from an area inside the casing to an area outside the casing, the exhausting occurring during at least a portion of a time period before the deactivating the sterilizing element.

13. The method of claim 9, wherein the actuating the sterilizing element includes emitting light at a wavelength of between about 100 nm to 300 nm.

14. The method of claim 9, wherein the deactivating the sterilizing element is in response to one of an expiration of a predetermined time period or moving the container from the second position to the first position.

15. An apparatus, comprising:
a case;
a drawer movably disposed within the case;
a first ultraviolet light source disposed within the case;
a second ultraviolet light source disposed within the case, the first ultraviolet light source and the second ultraviolet light source disposed opposite the drawer;
an actuator operatively coupled to and configured to activate the first ultraviolet light source and the second ultraviolet light source; and
an exhaust fan coupled to the case and configured to move air from an area inside the case to an area outside the case.

16. The apparatus of claim 15, wherein at least one of the first ultraviolet light source and the second ultraviolet light source emit a light at a wavelength in the range of between about 100 nm to 200 nm.

17. The apparatus of claim 15, wherein at least one of the first ultraviolet light source and the second ultraviolet light source emit a light at a wavelength of between about 200 nm to 300 nm.

18. The apparatus of claim 15, wherein the exhaust fan is configured to operate during at least a time period when the first ultraviolet light source and the second ultraviolet light source are actuated.

19. The apparatus of claim 15, wherein the first ultraviolet light source and the second ultraviolet light source emit light at different wavelengths.

20. An apparatus comprising:
a sterilizing element; and
a controller coupled to the sterilizing element, the controller configured to actuate the sterilizing element at a wavelength sufficient to sterilize currency.

21. The apparatus of claim 20, further comprising:
an actuator coupled to the controller, the actuator configured actuate the sterilizing element.

22. The apparatus of claim 20, wherein the sterilizing element includes at least one ultraviolet light source.

23. The apparatus of claim 20, wherein the sterilizing element includes at least one germicidal light source.

24. The apparatus of claim 20, wherein the sterilizing element includes at least one of a vacuum ultraviolet (VUV) light source, an ultraviolet-B (UV-B) light source or an ultraviolet-C (UV-C) light source.

25. The apparatus of claim 20, wherein the sterilizing element is configured to be actuated for a predetermined time period.

26. The apparatus of claim 20, wherein the sterilizing element is configured to emit light at a wavelength of between about 100 nm to 300 nm.

27. The apparatus of claim 20, further comprising:
an outer casing, the controller configured to be coupled to the outer casing; and
an inner container, the inner container configured to be movable relative to the outer casing, the sterilizing element configured to be positioned opposite the inner container.

28. The apparatus of claim 20, further comprising:
an outer casing, the controller configured to be coupled to the outer casing; and
an inner container, the inner container configured to move from a first configuration to second configuration, the inner container configured to actuate the controller in the second configuration.
